(19) United States
(12) Patent Application Publication
(10) Pub. No.: US 2009/0287133 A1
(43) Pub. Date: Nov. 19, 2009

(54) Disposable or Reclosable Wound or Skin Condition Dressing and Treatment System

(76) Inventor: Alfred J. LaGreca, SR., Hingham, MA (US)

Correspondence Address: MIRICK, O'CONNELL, DEMALLIE & LOUGEE, LLP, 1700 WEST PARK DRIVE, WESTBOROUGH, MA, 01581, US

(21) Appl. No.: 12/535,253

(22) Filed: Aug. 4, 2009

Related U.S. Application Data
(63) Continuation-in-part of application No. 11/749,833, filed on May 17, 2007, now Pat. No. 7,576,257.
(60) Provisional application No. 61/089,229, filed on Aug. 15, 2008.
(60) Provisional application No. 60/804,004, filed on Jun. 6, 2006.

(51) Int. Cl. A61L 15/44; A61F 13/02; A61H 7/00

(52) U.S. Cl. 602/54; 604/304; 601/84

(57) ABSTRACT

A reclosable wound or skin condition dressing and treatment system having a generally annular base portion having a circumference and lower and upper sides and defining an open volume within the circumference, the lower side carrying an adhesive that adheres to the skin and the upper side defining a release surface, a covering portion having a lower side that adhesively releasably engages with the release surface of the base portion, the covering portion fully covering the open volume of the base portion, to cover a wound or skin condition located in the open volume, and a pad coupled to the lower side of the covering portion and located within the open volume of the base portion.

### CROSS REFERENCE TO RELATED APPLICATIONS

This application claims priority of Provisional Patent Application Ser. No. 61/089,229, filed on Aug. 15, 2008. This application is also a Continuation In Part of and claims priority of application Ser. No. 11/749,833, filed on May 17, 2007, which itself claims priority of Provisional Patent Application Ser. No. 60/804,004 filed on Jun. 6, 2006. The disclosures of each of these prior applications are incorporated herein by reference.

### FIELD OF THE INVENTION

The invention relates to a wound or skin treatment dressing system.

### BACKGROUND OF THE INVENTION

Wounds are often covered with a surgical pad such as gauze while they are healing. Gauze is typically held in place with surgical tape. In order to inspect the wound or change the dressing pad, the tape must be removed and excess adhesive residue cleansed from the skin. For wounds that require frequent care and take days to heal, this constant tape removal process often irritates and damages the skin, especially for diabetics, the elderly and those with sensitive skin. This causes pain, and can lead to infections or other serious side effects.

### SUMMARY OF THE INVENTION

A purpose of the invention is to reduce the number of adverse events caused by the time required for nurses, doctors and patients to remove taped surgical pads from wounds for evaluation or for changing, as well as to reduce the negative factors related to the removing of the tape. The removal of tape, over the time required for wounds to heal, can cause scaring, infections and dermatitis. Further, the necessary repeated removal is very painful.

The invention has at least the following benefits:
Reduces hospital costs by reducing the time it takes a nurse or doctor to change a bandage. This reduces the effectiveness of the medical staff which in turn leads to adverse events such as infections, wrong medications etc. Thousands of patients die each year due to adverse events most caused by the shortage of medical personnel.
Reduces disposal of medical waste, including hazardous medical waste.
Increases home care patient independence.
Reduces unnecessary bandage changes as wound and dressing can be evaluated without removal of bandage.
May reduce infection from bacteria.
Reduces pain, scarring and dermatitis caused by the frequent changing of a bandage held to the skin with tape.
Allows bandage to be changed and wound to be evaluated or dressed as frequently as desired without irritation that is caused by tape removed from the skin.
Improves the quality of wound management as the type of dressing/medication may be changed without having to remove the bandage.
Decreases risk of skin conditions such as skin irritations, an example being bed sores, from becoming ulcers. This is because the skin condition may be treated prior to an ulcer or wound forming.

This invention features in one embodiment a disposable or reclosable wound or skin condition treatment or dressing system, comprising a base portion having a circumference and lower and upper sides, the lower side carrying an adhesive that adheres to the skin, the upper side defining a first part of a two-part mechanical or adhesive fastener system. The base portion defines an open area within the circumference. There is also a covering portion having a lower side that defines the second part of the two-part mechanical or adhesive fastener system. The covering portion overlies and engages with the upper side of the base portion. The covering portion also fully covers the open area of the base portion, to cover a wound or skin condition located in the open area. The disposable or reclosable dressing system may further comprise a removable protective member covering the adhesive on the lower side of the base portion.

The adhesive that adheres the base to the skin may comprise a butyl-based adhesive composition. The base portion preferably circumscribes the wound or the skin condition being treated. The covering portion may overlay essentially all of the base portion. The mechanical fastener system may comprise a two-part hook and loop fastener system, and the upper side of the base portion may define the hook part of the hook and loop fastener system. The reclosable dressing system may further comprise a wound or skin condition dressing pad located in the open area of the base portion. The dressing pad may be in contact with the lower side of the covering portion. The dressing pad may be coupled to the lower side of the covering portion by a polyurethane semi permeable breathable/waterproof membrane. This may be accomplished with an adhesive on the underside of the covering portion that is in contact with the dressing.

The reclosable dressing system may further accommodate a drainage tube which is secured proximate a wound, lies upon a dressing pad which absorbs excess fluids, and extends out from the base and covering portions to allow drainage of the wound.

The cover portion and/or the base portion may define a pull-tab adapted to be gripped by a user to assist in separating the cover portion from the base portion. The cover portion may have an upper side comprising an outer substantially impervious skin, which may be opaque.

This invention also features a reclosable wound or skin condition dressing and treatment system comprising a generally annular base portion having a circumference and lower and upper sides and defining an open volume within the circumference, the lower side carrying an adhesive that adheres to the skin and the upper side defining a release surface, a covering portion having a lower side that adhesively releasably engages with the release surface of the base portion, the covering portion fully covering the open area of the base portion, to cover a wound or skin condition located in the open volume, and a pad coupled to the lower side of the covering portion and located within the open volume of the base portion. The release surface of the base portion preferably comprises a material selected from the group of materials including plastics, hydrocolloids and foams.

The adhesive may comprise a butyl-based adhesive composition. The covering portion may overly essentially all of the upper side of the base portion. The pad may comprise a substance for treating a wound or a skin condition. The covering portion may define a pull tab adapted to be gripped by a user to assist in separating the covering portion from the base portion. The covering portion may have an upper side comprising an outer breathable and substantially waterproof membrane. The system may further comprise a removable protective cover over the adhesive on the lower side of the base portion. The covering portion and the pad may comprise an integral removable replaceable bandage.

The system may further comprise a drainage tube, feeding tube, IV tube or another tube or line coupled to the body. The tube is held in place by being sandwiched between the upper and lower portions. The tube or line extends out from the base portion and the covering portion, so that it is held in place for the desired function. The pad may comprise a gauze pad located in the open volume. The gauze pad may have a perimeter and define an open interior area that communicates with the perimeter. The tube or line may be located in the open area of the gauze pad.

The invention also features a reclosable wound or skin condition dressing and treatment system comprising a generally annular base portion having a circumference and lower and upper sides and defining an open volume within the circumference, the lower side carrying an adhesive composition that adheres to the skin and the upper side defining a release surface comprising a material selected from the group of materials including plastics, hydrocolloids and foams. There is a removable protective cover over the adhesive on the lower side of the base portion. There is also a covering portion having a lower side that adhesively releasably engages with the release surface of the base portion and an upper side comprising an outer breathable and substantially waterproof film, the covering portion fully covering the base portion, to cover a wound or skin condition located in the open volume, and a pad comprising a substance for treating a wound or a skin condition, the pad fixed to the lower side of the covering portion and located within the open volume of the base portion, wherein the covering portion and the pad comprise an integral removable replaceable bandage.

Further featured is a reclosable wound or skin condition dressing and treatment system, comprising a generally tubular compression stocking with an opening defined in it, wherein the opening is placed over a wound or skin condition to be treated, a base fastener member comprising the hook part of a two-part hook and loop-type mechanical fastening system, the base fastener member located on the compression stocking around at least a portion of the periphery of the opening, a covering portion having a lower side that defines the loop part of the mechanical fastening system and that mechanically engages with the base fastener member, the covering portion fully covering the opening in the compression stocking, to cover a wound or skin condition located in the open volume, and a pad coupled to the lower side of the covering portion and located within the opening in the compression stocking.

The base fastener member may be located around the entire periphery of the opening in the compression stocking. The pad may comprise a substance for treating a wound or a skin condition. The covering portion may define a pull tab adapted to be gripped by a user to assist in separating the covering portion from the compression stocking. The covering portion may have an upper side comprising an outer breathable and substantially waterproof film.

### BRIEF DESCRIPTION OF THE DRAWINGS

Other objects, features and advantages will occur to those skilled in the art from the following description of the preferred embodiments of the invention, and the accompanying drawings, in which:

FIG. 1 is top view of an embodiment of the device of the invention in place surrounding a wound or skin condition;

FIG. 2 is a cross-sectional view of the embodiment of the device of the invention shown in FIG. 1;

FIG. 3 is a partial, schematic, greatly enlarged cross-sectional view detailing the construction of a different embodiment of the invention;

FIG. 4 is a partial top view of yet another embodiment of the invention that is adapted to also hold a drainage tube in place proximate the wound or skin condition; and

FIG. 5 is a cross-sectional view of another embodiment of the invention that uses adhesive coupling of the top member to the base.

### DETAILED DESCRIPTION OF CERTAIN EMBODIMENTS OF THE INVENTION

This invention may be accomplished in a disposable or reclosable wound or skin condition dressing and treatment system. Essentially, the system comprises a two-part dressing, in which the part coupled to the skin (the base portion) remains on the skin during some or all of the course of treatment. The base portion carries on its lower side a long-term adhesive that adheres to the skin and can be left in place for about 5-7 days. The upper side of the base portion defines a first part of a two-part mechanical or adhesive fastener system. The base portion defines an open volume in which the wound is located. A covering portion has a lower side that defines the second part of the two-part mechanical or adhesive fastener system. The covering portion overlies and engages with the upper side of the base portion. The covering portion fully covers the open volume of the base portion, to cover a wound or skin condition located in the open area. In many but not all cases, a pad (for dressing or treatment) is coupled to the lower side of the covering portion and is located within the open volume of the base portion. The coupling can be a permanent fixation (such as with a bandage having an integral pad), or a mechanical or adhesive attachment of the pad to the underside of the covering portion, or the coupling can comprise the pad being held in place in the volume by the covering portion.

An embodiment of the inventive wound or skin condition dressing and treatment system 10 has at least two separable components: a base member 8 that is adhered to skin surface 32, and a cover or top member 9. The bandage, dressing pad or treatment pad itself can be part of cover member 9, or the invention can be used to hold a pad in place against the skin, or the invention can be used to cover the area of the skin without the use of a pad. The base member is an annulus or other shape that defines an open interior volume. The base member is semi-permanently adhered to the skin, with the wound or skin condition being treated located within the open volume such that the base surrounds the wound or skin condition, preferably without touching it. The top member typically either carries or holds in place, depending on the application, gauze, a dressing pad, a skin condition or wound treatment pad, or another type of wound or skin condition covering or other treatment modality. The base member provides the function of a means to removably couple the top member to the skin. Removable coupling can be accomplished by making the base member and the top member of the complementary mating portions of a hook and loop fastener system such as that sold by the 3M Company of St. Paul, Minn. or the Velcro Company of Manchester, N.H. Removable coupling of the base member and the top member can also be accomplished by an adhesive removable fastening system that has appropriate functionality, as further described below.

The adhesion of the base member to the skin may be accomplished with an adhesive that is compatible with skin. The adhesive may be that used on NEXCARE™ absolute waterproof first aid tape by the 3M Company (Minneapolis, Minn.), or a hydrocolloid adhesive such as that disclosed in U.S. Pat. No. 4,952,618, for example, or foam tape from 3M. These adhesives may remain in contact to the skin for five to seven days under normal use, including movement and exposure to water, without loosening and without substantial negative impact to the skin. In an embodiment of the invention, tape is not used and the base member remains on the skin for multiple days. This obviates the need for frequent removal of tape from the skin that leads to so many problems in the current wound-care regimen.

The base can have a desired, preferably annular shape, such as a circular, oval or rectangular annulus, for example. A certain embodiment of the base member 8 comprises a unitary construction of a strip of waterproof adhesive 14 approximately three quarter of an inch wide secured to the entire underside of an annulus 12 of the hook side of a VELCRO™ type fastener system. The adhesive is used to secure the base to the person's skin, surrounding the wound or area of the skin requiring covering or treatment. Adhesives that can be used to couple the base to the skin are hydrocolloid material such as 3M #9943 or #9944, or DUODERM™ by Convatec of Skellman, N.J., or an adhesive hydrogel such as available from Scapa North America in Windsor, Conn. The adhesive would normally be covered with a removable protective layer 16 as is commonly used to protective the sticky side of an adhesive tape, label or sticker. The base is preferably sterile, and can be made with anti-bacterial material.

Top member 9 is typically the same size and perimeter shape as the base member, except that it is not annular. Accordingly, member 9 entirely covers base member 8, and also spans the opening 34 defined by the base member, to enclose the volume in which wound or skin condition 30 is located. Member 9 also encloses dressing or treatment pad 24, should such be used. In this embodiment, top member 9 can be constructed in at least two manners. In one manner, member 9 is a piece of fabric that defines loops that are adapted to mechanically engage with the hooks of the base member. In a second manner of construction, the top member comprises an annulus 18 of the same size and shape as the base member, and defines the loop construction. In this case, top member 9 includes a semi permeable membrane 20 of polyurethane such as 3M™ #9833 that is secured to the top portion 18 and spans the annular opening of portion 18. Sheet 20 can have an adhesive on its underside so that the loop annulus can be carried by the sheet, and if desired the dressing or other treatment pad or the like can be adhered to the underside of the top sheet. This way, the inventive system can be used with or without a treatment pad or any other wound or skin-care material located in the volume bounded by the bottom and top members.

Top member 9 is sterile and can be made of anti-bacterial material. Member 9 may carry a dressing or treatment pad 24, in which case top member 9 is disposed of when pad 24 is changed. Alternatively, sheet 20 may be used to enclose the volume in which pad 24 is located, to hold the pad in place covering the wound, skin condition, or area of skin that is to be enclosed by the inventive device: this allows the pad to be disposed of and replaced as needed, while retaining the base member 8. In this case another dressing pad or the like is inserted over the wound or skin condition and sealed in place by pressing top member 9 to the base member 8.

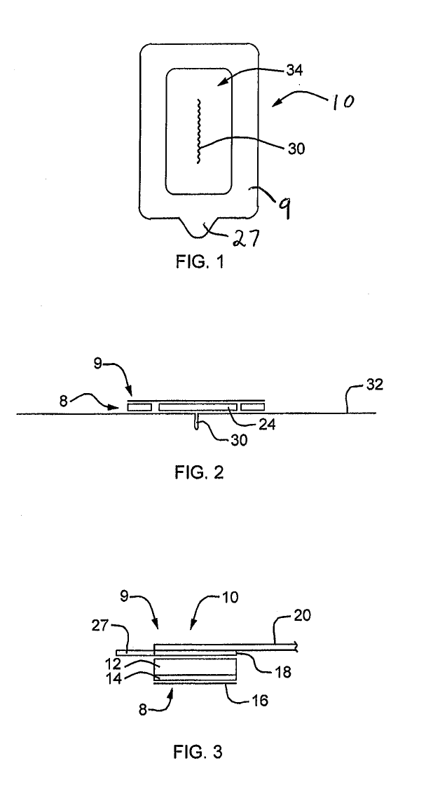

The exact configuration of the inventive system depends at least on one or more of the type of wound or skin condition, the specific dressing or treatment pad or the like (if required), and the expected frequency with which the top will be removed or peeled back from the base. Sheet 20 can be opaque or translucent, waterproof, breathable, and/or hydrophobic, depending upon the material from which it is constructed. Sheet 20 can be a medical grade polyurethane semi-permeable membrane that provides a waterproof breathable cover over the skin region but not touching it. If the top is translucent (as is the case in FIG. 1) the pad or the underlying skin region can be inspected without separating the top member from the bottom member.

The top member can comprise a layer of a knit polyester or nylon fabric having loops that act as the loop portion of a hook and loop fastener system. The base member can be made from an extruded polypropylene or knit nylon, for example. Both are available from Aplix, Inc. of Charlotte, N.C. (#N35 loop and #946 hook) or 3M™ (#7331 or #7333) or VELCRO™ (#3008). In the case in which sheet 20 is used, it may be made from TEGADERM™ film available from 3M, which is a breathable, waterproof layer which also provides a barrier to outside contaminants. Sheet 20 can carry an adhesive on its underside so that both the annulus 18 of loop-defining material and the wound care or skin treatment pad can be carried by it.

Bottom member 8 and top member 9 may each have a projecting tab such as tab 27. The tabs provide an area that can be grasped by the user to hold down the base and provide for the easy release of the top member from the base member in order to access the wound.

The inventive system is a very low-profile two-part wound covering that can stay in place for many days, thus decreasing the need to pull tape off of the skin in order to change a bandage or treatment pad. The result is less patient pain and suffering, and decreased costs associated with treating infections caused by repeated tape removal, which is prevalent in chronic-care situations in which a dressing may need to be changed multiple times per day for a week or more. This also decreases medical costs by changing dressings only when necessary, decreased waste and decreased labor costs associated with removing tapes and their residue. Increasing nursing and medical availability caused by this invention will decrease adverse events causing complications and sometimes death.

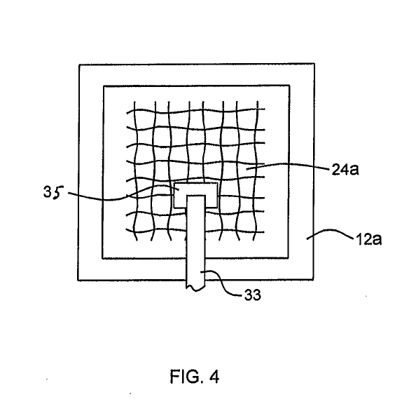

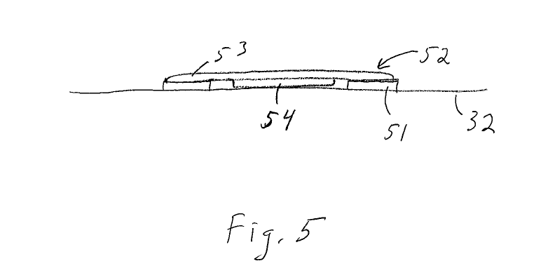

FIG. 4 shows the base member and dressing pad of another embodiment that is adapted to also hold a wound drainage tube, IV tube or other patient treatment tube or line in place attached within the body and extending from the wound. Base member 12a has a slit in one side so that it can be slipped under tube 33. Dressing pad 24a has void area 35 to accommodate tube 33 where it emerges from the skin, and is placed around and under tube 33, to absorb excess fluid. A slot cut in pad 24a allows the pad to be tucked under the tube. An opening or slit in the cover member (not shown) can be included to allow for tube 33 to project through the inventive dressing system and be extended to a vessel or other external equipment. The top member (not shown) is sufficiently flexible such that it can be attached to annulus 12 at all locations except where the top overlies tube 33 as it crosses over annulus 12a. The flexibility of the top member allows a tight enough fit over tube 33 such that it is held in place in the interior of the inventive system.

The inventive system contemplates a variety of means for coupling the base member to the skin of a person or animal, a variety of means for removably coupling the top member to the base member, and a variety of wound and skin condition treatment modalities held in place or enabled by the base and top members.

For example, the base can be coupled to the skin with any adhesive that accomplishes sufficient adhesion along with the benefits of less skin irritation or damage, such as a TEGADERM™ adhesive from 3M. An additional example is adhesive silicone gels such as those manufactured and used by Scapa, Windsor, Conn. Also, the inventive dressing can be removably coupled to the skin by an elastic compression wrap bandage overlaying the inventive dressing (such as an ACE™ bandage from BD, Franklin Lakes, N.J.) and wrapped around a portion of the body such as a limb or the torso.

Another means to effectively accomplish a base member would be to provide an open area in an essentially tubular compression stocking, and line most or preferably all of the perimeter of the opening with a base fastener member comprising the hook portion of a hook and loop fastener system. Then, the stocking can be applied to a limb, with the opening located over the area to be treated. One of the top member embodiments that carries loop fasteners can then be applied over the opening, mechanically coupled to the base fastener member. In this manner, no adhesive of any sort is necessary on the base.

FIG. 5 shows another embodiment. In this case, top 52 is coupled to annular base 51 by an adhesive, such as one of the adhesives set forth above. Top 52 can comprise an adhesive-coated film that carries pad 54. One example of top 52 is an adhesive bandage such as BIATAIN™ 3430 adhesive bandage by Coloplast Corp, Minneapolis, Minn., or 3M TEGADERM™ adhesive bandage. The adhesive that couples the top to the base needs to be sufficient to hold the top on the base but allow the top to be removed and placed back on the base at least once, and preferably multiple times, over the up to 5-7 day typical useful life of the inventive device. This adhesive needs to be weaker than the adhesive that holds the base on the skin so as not to disturb the base. One example of an adhesive that may be useful for the top member is a medical grade pressure sensitive tape adhesive such as 3M™ #1510 on the top member and a plastic (polyethylene) upper surface on the base member. Release surfaces for the top of the bottom portion of the inventive system are preferably accomplished by the use of a plastic layer, a hydrocolloid, or a foam. In one embodiment, the bottom portion is an annular piece of appropriate medical-grade hydrocolloid or foam, with adhesive on one side (that rests against the skin) and no adhesive on the other side (so that the adhesive top portion can be removably or releasably coupled to the top surface of the bottom portion).

There are many examples of substances that can make up or be carried by the pad, gauze or the like that is held in place by the top member, lies against the skin, and/or protrudes into a wound in the skin. As the inventive system can be opened and reclosed, and allows the top to be replaced, the invention can be use to accomplish a desired treatment regimen for a wound, skin condition, or the like. As one example, the pad can carry or comprise or be entirely made from certain known materials that have known medical effects. Among these are:
Foam composites such as MEPILEX™ Soft Silicon by Molnlycke of Denmark. These products typically serve more than purpose, for example a combination which maintains moisture in the wound site, is a barrier to bacteria, and absorbs excess exudates.
Alginates such as ALGICELL™ Calcium Alginate by Derma Sciences of Princeton, N.J. These absorb excess exudates, maintain a moist healing environment, and facilitate autolytic debridement.
Silver alginate such as MAXSORB EXTRA™ by Medline Industries of Mundelsein, Ill. In addition to the benefits of the alginate, the silver reduces infections from a wide range of microorganisms.
Antimicrobial such as ANTICOAT 7™ by Smith and Nephew of London, England. This can be an effective barrier to bacterial penetration that may reduce infections in partial or full thickness wounds.
Composites such as THINSITE HYDROGEL™ by Swiss America of Waldenburg, Switzerland. These are made of a combination of wound dressings which improve healing, reduce infections and absorb exudates.
Transparent dressings such as TEGADERM™ #1628 by 3M allow the wound to be viewed at all times but have the disadvantage of difficulty in removal from the skin and wound. This significantly reduces the damage of removal but allows for continued viewing of the wound or skin condition or the like.
Hydrocolloids such as DERMATELL™ by Gentell, Inc of Skellman, N.J. These support a moist healing environment and autolytic debridement and non traumatic dressing removal.
Hydrogels such as AMERIDERM™ wound gel dressing by Ameriderm Labs Ltd. Of Patterson, N.J. The gel's high glycerin content facilitates the natural wound healing process.
Silver such as SILVERLON™ by Argentum Medical LLC of Willowbrook, Ill. reduces infections by resisting and eliminating most microorganisms.

Of course, the invention contemplates other materials, medications and the like, to accomplish any desired or custom treatment of a condition such as skin irritation caused by pressure prior to the wound developing, or a surface wound. The invention speeds and eases wound and skin care and treatment in situations in which the skin needs to be covered. The invention also decreases incidence of tape removal-related infections. The invention thus can reduce the costs associated with wound and skin care.

### Example 1

The sterilized base is removed from its sterile packaging and is attached to the skin approximately one inch away from and surrounding the incision, cut or wound to be protected, or skin condition to be treated. The top is removed from its sterile packaging. The top carrying the dressing or treatment pad is placed over the wound or the area of the skin being treated, aligning the top with the base. Light pressure to the top engages the hook and loop material to ensure a tight fit, and potentially a tight, waterproof or water resistant dressing.

Periodically (normal dressing change would be each day; chronic up to four times per day) the top is removed from the base by holding the base in place using tab 27 while pulling gradually on the tab fixed to the top. The edges of the exposed area of skin can then be dabbed with disinfectant or another topically applied skin treatment, and a new top carrying a dressing or treatment pad or the like is applied to the base.

### Example 2

The base member and top member are removed from their sterile package. They can be arranged in a clamshell-type arrangement with the members attached together at one end. The top is separated from the bottom by use of the tab, and the appropriate dressing or pad is applied. Periodically (normally dressing change would be each day; chronic up to four times per day) the top is peeled back from the base and a new dressing or pad is applied. Both the base member and the top member are re-used during the entire course of treatment. If there is excessive drainage, the top may be replaced if necessary.

Although specific features of the invention are shown in some figures and not others, this is for convenience only, as some features may be combined with any or all of the other features in accordance with the invention.

Recitation of ranges of values herein are merely intended to serve as a shorthand method of referring individually to each separate value falling within the range, unless otherwise indicated herein, and each separate value is incorporated into the specification as if it were individually recited herein.

The use of any and all examples, or exemplary language (e.g., “such as”) provided herein, is intended merely to better illustrate the invention and does not pose a limitation on the scope of the invention.

A variety of modifications to the embodiments described herein will be apparent to those skilled in the art from the disclosure provided herein. Thus, the invention may be embodied in other specific forms without departing from the spirit or essential attributes thereof.

What is claimed is:

1. A reclosable wound or skin condition dressing and treatment system, comprising:
a generally annular base portion having a circumference and lower and upper sides and defining an open volume within the circumference, the lower side carrying an adhesive that adheres to the skin and the upper side defining a release surface;
a covering portion having a lower side that adhesively releasably engages with the release surface of the base portion, the covering portion fully covering the open volume of the base portion, to cover a wound or skin condition located in the open volume; and
a pad coupled to the lower side of the covering portion and located within the open volume of the base portion.

2. The system of claim 1 wherein the adhesive carried by the lower side of the base portion comprises a butyl-based adhesive composition.

3. The system of claim 1 wherein the covering portion overlies essentially all of the upper side of the base portion.

4. The system of claim 1 wherein the pad comprises a substance for treating a wound or a skin condition.

5. The system of claim 1 wherein the covering portion defines a pull tab adapted to be gripped by a user to assist in separating the covering portion from the base portion.

6. The system of claim 1 wherein the covering portion has an upper side comprising an outer breathable and substantially waterproof membrane.

7. The system of claim 1 further comprising a removable protective cover over the adhesive on the lower side of the base portion.

8. The system of claim 1 wherein the covering portion and the pad comprise an integral removable replaceable bandage.

9. The system of claim 1 further comprising a tube or line in contact with the lower side of the covering portion and extending out from the base portion and the covering portion, to hold the drain or line in place relative to the body.

10. The system of claim 9 wherein the pad comprises a gauze pad located in the open volume.

11. The system of claim 10 wherein the gauze pad has a perimeter and defines an interior open area that communicates with the perimeter.

12. The system of claim 11 wherein the tube or line is located in the open area of the gauze pad.

13. The system of claim 1 in which the release surface of the base portion comprises a material selected from the group of materials including plastics, hydrocolloids and foams.

13. A reclosable wound or skin condition dressing and treatment system, comprising:
a generally annular base portion having a circumference and lower and upper sides and defining an open volume within the circumference, the lower side carrying an adhesive composition that adheres to the skin and the upper side defining a plastic, hydrocolloid or foam-based release surface;
a removable protective cover over the adhesive on the lower side of the base portion;
a covering portion having a lower side that adhesively releasably engages with the release surface of the base portion and an upper side comprising an outer breathable and substantially waterproof film, the covering portion fully covering the base portion, to cover a wound or skin condition located in the open volume; and
a pad comprising a substance for treating a wound or a skin condition, the pad fixed to the lower side of the covering portion and located within the open volume of the base portion, wherein the covering portion and the pad comprise an integral removable replaceable bandage.

14. A reclosable wound or skin condition dressing and treatment system, comprising:
a generally tubular compression stocking with an opening defined in it, wherein the opening is placed over a wound or skin condition to be treated;
a base fastener member comprising the hook part of a two-part hook and loop-type mechanical fastening system, the base fastener member located on the compression stocking around at least a portion of the periphery of the opening;
a covering portion having a lower side that defines the loop part of the mechanical fastening system and that mechanically engages with the base fastener member, the covering portion fully covering the opening in the compression stocking, to cover a wound or skin condition located in the open volume; and
a pad coupled to the lower side of the covering portion and located within the opening in the compression stocking.

15. The system of claim 14 wherein the base fastener member is located around the entire periphery of the opening in the compression stocking.

16. The system of claim 14 wherein the pad comprises a substance for treating a wound or a skin condition.

17. The system of claim 14 wherein the covering portion defines a pull tab adapted to be gripped by a user to assist in separating the covering portion from the compression stocking.

18. The system of claim 14 wherein the covering portion has an upper side comprising an outer breathable and substantially waterproof film.
